(19) United States
(12) Patent Application Publication
(10) Pub. No.: US 2013/0281914 A1
(43) Pub. Date: Oct. 24, 2013

(54) TRANSDERMAL DRUG ADMINISTRATION DEVICE

(71) Applicant: Mitsutoshi Yaegashi, Ashigarakami-gun (JP)

(72) Inventor: Mitsutoshi Yaegashi, Ashigarakami-gun (JP)

(73) Assignee: TERUMO KABUSHIKI KAISHA, Shibuya-ku (JP)

(21) Appl. No.: 13/996,384

(22) PCT Filed: Dec. 19, 2011
(86) PCT No.: PCT/JP2011/079335

§ 371 Date: Jun. 20, 2013

(30) Foreign Application Priority Data
Dec. 28, 2010 (JP) 2010-292842

(51) Int. Cl. A61N 1/30

(52) U.S. Cl. CPC A61N 1/303
USPC 604/20

(57) ABSTRACT

A transdermal drug administration device which comprises a patch that is provided with: a donor gel in which a medical agent is sealed; a reference gel; a first electrode that is connected to the donor gel; and a second electrode that is connected to the reference gel. When the patch is applied to the skin, a direct current is applied between the first electrode and the second electrode at a predetermined cycle and an alternating current is applied between the first electrode and the second electrode during the periods when a direct current is not applied, thereby obtaining the capacitance. The application state of the patch is determined based on the thus-obtained capacitance.

### TECHNICAL FIELD

The present invention relates to a transdermal drug administration device using the principle of iontophoresis that allows a medical agent to infiltrate a skin of a human being by passing a low electric current through the skin.

### BACKGROUND ART

Transdermal drug administration methods include an iontophoresis. The iontophoresis is a method in which positive and negative electrodes are attached to respective two separated points on a skin, and an electric current is passed from one of the electrodes across the stratum corneum to the other electrode to thereby move a charged drug on the basis of the principle of electrophoresis, whereby transdermal drug absorption is facilitated. In this case, the current is basically constant. The area of each of the electrodes is also constant. Therefore, the current density, which is current per unit area, is also constant.

One of the positive and negative electrodes is in contact with a gel containing a drug, and referred to as a donor portion. The other electrode is in contact with a gel containing salt solution, and referred to as a reference portion.

In principle, the charged drug is subjected to facilitation of absorption. However, it has been reported that since a water flow is also generated by the flowing electric current, even a non-charged drug or even a high-molecular-weight drug exhibits increased skin permeability.

A common drug administration system using iontophoresis has a patch which contains a drug, and a controller which applies electric current to the patch. Conventionally, a drug administration system has generally utilized a method in which electric current is supplied by a commercial power supply (AC 100 V, for example). However, recently, a portable drug administration system that uses a battery (a coin type battery, for example) to supply electric current so as not to restrict the activities of a patient even during the administration, has been commercialized.

Japanese Laid-Open Patent Publication No. 2001-120669 described below discloses an iontophoresis device which detects abnormal conductivity at the beginning of energization and during energization. More specifically, it discloses that an output operation and an abnormal conductivity detecting operation are alternately switched in an output section, and when the device comes off, the electric energy supply is determined as abnormal by an abnormal conductivity detecting section, and the output of electric current is then stopped.

Japanese Laid-Open Patent Publication No. 2000-237330 described below discloses a device for iontophoresis capable of highly accurately determining a conduction state. More specifically, it discloses that the conduction state is determined as normal when the reactive current flowing in the skin is higher than a threshold level, and is determined as abnormal when the reactive current flowing in the skin is lower than the threshold level.

### SUMMARY OF INVENTION

However, in the above techniques described in Japanese Laid-Open Patent Publication Nos. 2001-120669 and 2000-237330, when the patch partially comes off the skin, and the contact area between the patch and the skin thereby decreases, although it is possible to stop energization, it is not possible to continue energization while taking into account the decrease of the contact area. On the other hand, if energization is merely continued when the contact area decreases, the applied current density increases with decreasing contact area, which causes an adverse effect on the skin of a patient.

Therefore, the present invention has been made in view of the above conventional problem, and an object thereof is to provide a transdermal drug administration device capable of continuing energization without causing an adverse effect on a skin even when the contact area between a patch and the skin varies.

In order to achieve the above object, the present invention according to claim 1 provides a transdermal drug administration device that includes a patch provided with a donor portion having a first contact member containing therein a medical agent, a reference portion having a second contact member, a first electrode connected to the first contact member, and a second electrode connected to the second contact member; and a control unit for controlling supply of an electric current to the first electrode. By applying the patch to an external conductor, the first contact member and the second contact member come into contact with the external conductor, and the first electrode and the second electrode are thereby electrically connected to the external conductor. When the patch is applied to the external conductor, the control unit supplies a direct current between the first electrode and the second electrode at a predetermined cycle, and also supplies an alternating current between the first electrode and the second electrode during a first period in which a direct current is not supplied, thereby obtaining a capacitance between the first electrode and the second electrode. By obtaining the capacitance between the first contact member and the second contact member in this manner, it is possible to obtain the contact area between the first and second contact members and the external conductor. This makes it possible to supply a direct current such that the density of the current flowing in the external conductor such as the skin of a patient is kept constant. As a result, even when, for example, the patch partially comes off the skin with the lapse of time, and the contact area thereby decreases, it is possible to continue energization without causing an adverse effect on the skin of a patient.

The present invention according to claim 2 provides the transdermal drug administration device according to claim 1, wherein the control unit obtains an impedance between the first electrode and the second electrode during the first period, obtains a resistance between the first electrode and the second electrode during a second period in which a direct current is supplied, and obtains the capacitance from the impedance and the resistance. This makes it possible to accurately obtain the capacitance.

The present invention according to claim 3 provides the transdermal drug administration device according to claim 1 or 2, the device further including a first circuit for supplying a direct current between the first electrode and the second electrode; a second circuit for supplying an alternating current between the first electrode and the second electrode; and a switch for switching connection of the first electrode and the second electrode between connection to the first circuit and connection to the second circuit, wherein the control unit controls the switch to connect the first electrode and the second electrode to the second circuit during the first period, and connect the first electrode and the second electrode to the first circuit during the second period in which a direct current is supplied. This makes it possible to selectively supply a direct current and an alternating current to the external conductor.

The present invention according to claim 4 provides the transdermal drug administration device according to any one of claims 1 to 3, wherein the control unit changes the value of a direct current to be supplied between the first electrode and the second electrode depending on the obtained capacitance to keep the density of the direct current to be supplied to the external conductor constant. Accordingly, even when a part of the patch comes off the external conductor, the density of the current flowing in the external conductor can be kept constant. As a result, it is possible to continue the energization without causing an adverse effect on the skin of a patient.

The present invention according to claim 5 provides the transdermal drug administration device according to any one of claims 1 to 4, wherein the control unit stops the supply of direct current between the first electrode and the second electrode if the obtained capacitance is equal to or lower than a predetermined value. This makes it possible to prevent an adverse effect on the skin of a patient due to excessive current density. Further, for example, in a case where the medical agent is an anesthetic such as lidocaine, if the contact area is smaller than the minimum area required for puncture, the supply of current is stopped. Therefore, it is possible to prevent the medical agent from being wastefully administered.

The present invention according to claim 6 provides the transdermal drug administration device according to any one of claims 1 to 5, wherein the control unit issues an alert if the obtained capacitance is equal to or lower than a predetermined value. Accordingly, for example, in a case where the medical agent is an anesthetic such as lidocaine, if the contact area is smaller than the minimum area required for puncture, an alert is issued. Therefore, a patient or a medical professional can recognize that a part of the patch has come off the external conductor, and the medical agent cannot therefore be appropriately administrated.

According to the present invention, a direct current is supplied between the first electrode and the second electrode at a predetermined cycle, and an alternating current is supplied between the first electrode and the second electrode during the first period in which a direct current is not supplied, whereby the capacitance between the first electrode and the second electrode is obtained. Therefore, it is possible to obtain the contact area between the first and second contact members and the external conductor. As a result, it is possible to supply a direct current to the external conductor (the skin, for example) such that the density of the direct current is kept constant. Therefore, even when the contact area decreases, it is possible to continue the energization without causing an adverse effect on the skin of a patient.

### BRIEF DESCRIPTION OF DRAWINGS

FIG. 1 is a perspective view illustrating the overall structure of a transdermal drug administration device using iontophoresis of an embodiment;

FIG. 2 is an exploded perspective view of an iontophoresis patch shown in FIG. 1;

FIG. 3 is a cross-sectional view of an electrode film taken along line III-III in FIG. 2;

FIG. 4A is a top plan view of the electrode film;

FIG. 4B is a bottom plan view of the electrode film;

FIG. 5 is an explanatory side view illustrating the transdermal drug administration device, shown in FIG. 1, arranged in close contact with an arm of a patient;

FIG. 6 is a circuit diagram of the transdermal drug administration device shown in FIG. 1 in a state where the patch of the transdermal drug administration device is applied to a skin;

FIG. 7 is a diagram illustrating an energization current table which is stored in a storage unit shown in FIG. 6;

FIG. 8 is a flowchart illustrating the operation of a control unit shown in FIG. 6;

FIG. 9 is a diagram illustrating change with time of the resistance between electrodes when electric current is passed through the skin using the principle of iontophoresis;

FIG. 10 is a perspective view illustrating the overall structure of a transdermal drug administration device of Modification 5;

FIG. 11 is an exploded perspective view of an iontophoresis patch shown in FIG. 10;

FIG. 12 is a top plan view of an electrode film;

FIG. 13 is a diagram illustrating an example of an arrangement relationship between holes and magnetic bodies in Modification 9;

FIG. 14 is a diagram illustrating another example of the arrangement relationship between the holes and the magnetic bodies in Modification 9; and

FIG. 15 is a perspective view of a transdermal drug administration device in Modification 10.

### DESCRIPTION OF EMBODIMENTS

A transdermal drug administration device using iontophoresis according to the present invention will be described in detail below by giving a preferred embodiment with reference to the appended drawings.

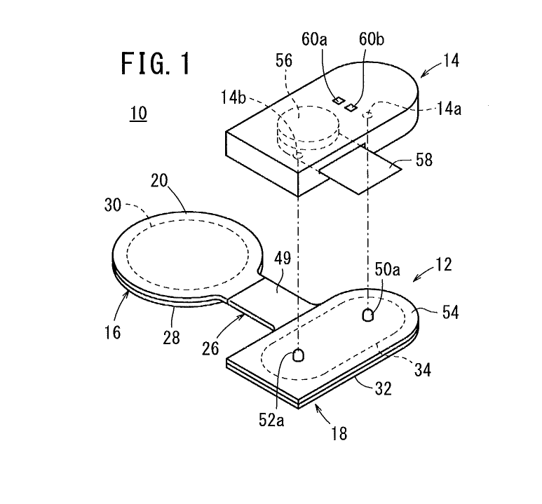

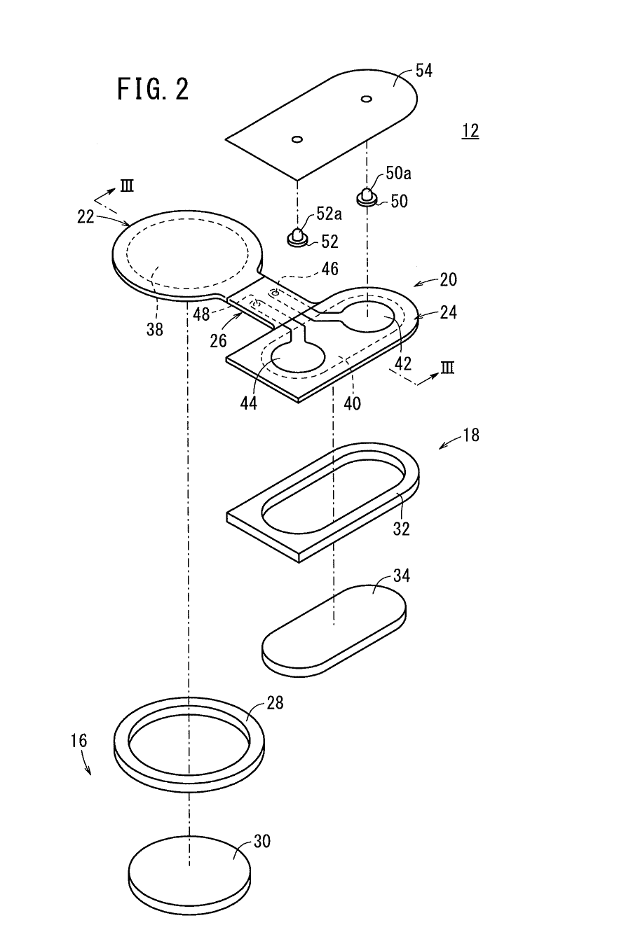

FIG. 1 is a perspective view illustrating the overall structure of a transdermal drug administration device 10. In FIG. 1, an iontophoresis patch 12 and an energization device 14 which constitute the transdermal drug administration device 10 are illustrated as being separated from each other. FIG. 2 is an exploded perspective view of the iontophoresis patch 12 shown in FIG. 1.

The transdermal drug administration device 10 (hereinafter, referred to as the device 10) is a medical instrument that is used to, for example, eliminate pain caused by puncture in a hemodialysis patient, and to administer and infiltrate a local anesthetic (an ionic anesthetic containing lidocaine, for example) into an arm of a patient. In the device 10, the iontophoresis patch 12 (hereinafter, referred to as the patch 12) is applied to the skin of the patient, which serves as an external conductor, and is energized by the energization device 14 to thereby allow the ionic anesthetic enclosed in the patch 12 to infiltrate into the living body. The patch 12 may be applied to a device for administering an ionic medical agent other than an ionic anesthetic to a patient, and may also be applied to a device for administering a medical agent other than an ionic one to a patient.

As shown in FIGS. 1 and 2, the device 10 is provided with the patch 12 and the energization device 14 which is placed on and connected to the surface (the upper surface) of the patch 12.

The patch 12 is provided with a donor portion 16 in the form of a circular thin sheet, and a reference portion 18 in the form of a rectangular thin sheet with an arched side, the reference portion 18 being separated from the donor portion 16. An electrode film (an electrode body) 20, to which the energization device 14 is connected, is provided across the donor portion 16 and the reference portion 18. The electrode film 20 includes a donor-side region 22 having a shape corresponding to the shape of the donor portion 16, a reference-side region 24 having a shape corresponding to the shape of the reference portion 18, and a narrow bridge region 26 which connects the donor portion 16 and the reference portion 18 to each other (see FIG. 2).

The donor portion 16 includes a donor application member 28 having a circular shape corresponding to the outer shape of the donor portion 16, and a donor gel (a first contact member) 30 with which to load an opening of the donor application member 28. The donor-side region 22 of the electrode film 20 is electrically connected to the surface (the upper surface in FIG. 2) of the donor gel 30. The reference portion 18 includes a reference application member 32 having a rectangular shape with an arched side substantially corresponding to the outer shape of the reference portion 18, and a reference gel (a second contact member) 34 with which to load an opening of the reference application member 32. The reference-side region 24 of the electrode film 20 is electrically connected to the surface of the reference gel 34.

Each of the donor application member 28 and the reference application member 32 is an adhesive elastic body which adheres to the skin of a human body or the like with a certain strength, and has an electrical insulation property. The ionic anesthetic is contained in the donor gel 30. A solvent or solution of an electrolyte (buffer salt or common salt, for example), which has no harmful effect on the living body, such as salt solution is contained in the reference gel 34. A medical professional having sufficient puncture skills can easily insert a needle into a puncture area of 2.5 cm² within which a medical agent is delivered. Therefore, for example, the area of the contact surface (lower surface in FIG. 2) of each of the donor gel 30 and the reference gel 34, for contact with the skin, should be approximately 2.5 to 5.0 cm².

The opening of the donor application member 28 is loaded with the donor gel 30, the opening of the reference application member 32 is loaded with the reference gel 34, and the donor application member 28 and the reference application member 32 are then applied to the skin of a patient. Accordingly, the donor portion 16 and the reference portion 18 can be brought into contact with the skin substantially at the same time. Therefore, the patch 12 can be easily applied to the skin with a single action. Further, the contact surfaces of the donor gel 30 and the reference gel 34 for contact with the skin may have adhesiveness.

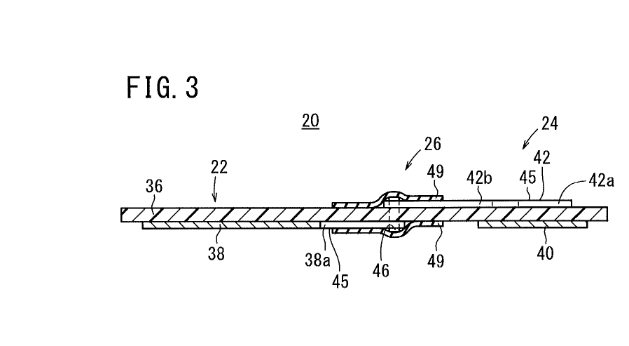

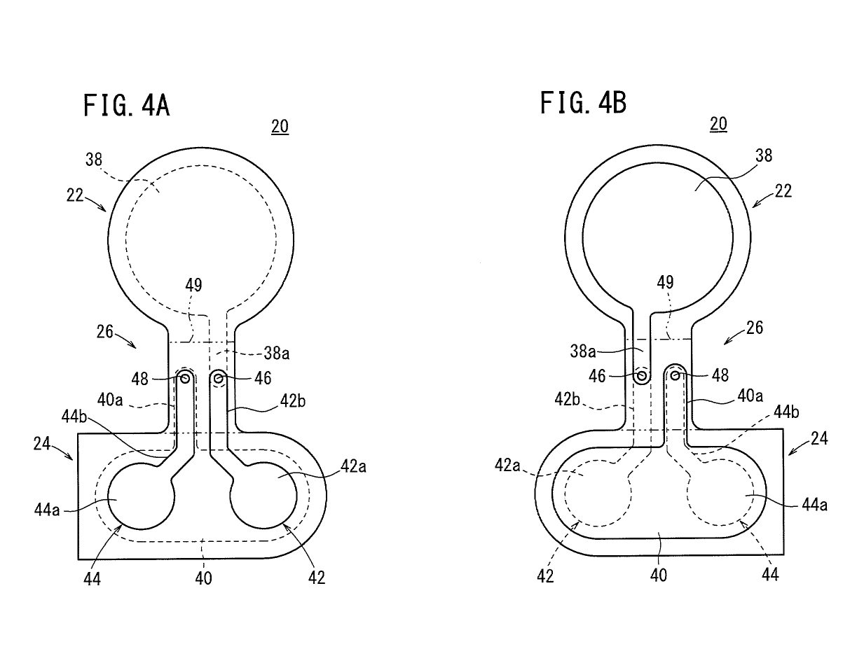

FIG. 3 is a cross-sectional view of the electrode film 20 taken along line III-III in FIG. 2. For easy understanding, the thickness of the electrode film 20 is illustrated in an exaggerated manner. FIG. 4A is a top plan view of the electrode film 20. FIG. 4B is a bottom plan view of the electrode film 20.

The electrode film 20 is a flexible printed circuit board having a flexible base 36 which defines the outer shape of the electrode film 20. Respective parts of the base 36 are defined as the donor-side region 22, the reference-side region 24, and the bridge region 26. The base 36 is, for example, a flexible film produced by forming a resin such as polyester, polyimide, or the like into a thin film-like shape.

As shown in FIGS. 3, 4A, and 4B, the donor-side region 22 is equipped with a circular first electrode 38 disposed on the bottom surface of the base 36, the first electrode 38 making contact with and being thereby electrically connected to the donor gel 30. A connection line 38a extends from the first electrode 38 up to a substantially central portion of the bridge region 26 along one side of the bridge region 26.

The reference-side region 24 includes an oblong second electrode 40 disposed on the bottom surface of the base 36, the second electrode 40 making contact with and being thereby electrically connected to the reference gel 34. A pair of contact terminal lines (a first contact terminal line and a second contact terminal line) 42 and 44 is juxtaposed on the surface of the base 36. A connection line 40a extends from the second electrode 40 up to a substantially central portion of the bridge region 26 along the other side of the bridge region 26 in parallel to the connection line 38a (see FIG. 4B). The contact terminal lines 42 and 44 have respective circular terminal bases 42a and 44a which are paired and juxtaposed on the surface of the reference-side region 24, and also have respective connection lines 42b and 44b which are paired and extend from the respective terminal bases 42a and 44a and bent up to the substantially central portion of the bridge region 26 in parallel to each other.

The interconnections constituting the electrode film 20, that is, the first electrode 38 (the connection line 38a), the second electrode 40 (the connection line 40a), and the contact terminal lines 42 and 44 (the connection lines 42b and 44b, and the terminal bases 42a and 44a) are formed, for example, by printing an electrically conductive ink containing silver or silver/silver chloride on the front surface and the back surface of the base 36. Exposed surfaces of the interconnections are sealed by, for example, an insulating resin material 45 (see FIG. 3). However, the resin material 45 is not applied to the bottom surfaces (the contact surfaces) of the first electrode 38 and the second electrode 40 which respectively make contact with the donor gel 30 and the reference gel 34. Further, the electrically conductive ink may be printed a plurality of times to increase its thickness so that the occurrence of conduction failure can be more reliably prevented.

As shown in FIGS. 3, 4A, and 4B, the connection line 38a of the first electrode 38 and the connection line 42b of one of the contact terminal lines (the first contact terminal line 42) are aligned with each other in the thickness direction of the bridge region 26 (see FIGS. 3 and 4B). Distal ends of the connection line 38a and the connection line 42b are electrically connected to each other by a through hole 46 which penetrates the bridge region 26 in the thickness direction thereof. Similarly, the connection line 40a of the second electrode 40 and the connection line 44b of the other contact terminal line (the second contact terminal line 44) are aligned with each other in the thickness direction of the bridge region 26. Distal ends of the connection line 40a and the connection line 44b are electrically connected to each other by a through hole 48 which penetrates the bridge region 26 in the thickness direction thereof.

As described above, in the electrode film 20, the base 36 of the donor-side region 22 has a one-sided interconnect structure having only the first electrode 38. The base 36 of the reference-side region 24 has a double-sided interconnect structure having the second electrode 40, and the contact terminal lines 42 and 44. Therefore, in the patch 12, the donor portion 16 which is provided with the donor-side region 22 having the one-sided interconnect structure is more flexible than the reference portion 18 which is provided with the reference-side region 24 having the double-sided interconnect structure. Further, the bridge region 26 has the interconnections and the through holes 46 and 48 concentrated in a substantially central portion thereof. However, the bridge region 26 has the one-sided interconnect structure having only the connection line 38a near the donor-side region 22. Therefore, the bridge region 26 also has a large flexibility near the donor portion 16.

As shown in FIGS. 1 and 3, the bridge region 26 is surrounded by cover lays (protection layers) 49 each of which comprises an insulating sheet material in order to prevent the connection lines 38a, 40a, 42b, 44b, and the through holes 46, 48 from being exposed to the outside. Insulating coatings (resist layers) or the like may be used instead of the cover lays 49. However, when taking the flexibility and the durability in the bridge region 26 into consideration, the cover lays 49 in the form of sheet materials are effective.

As shown in FIG. 2, connection terminals (hooks) 50, 52 are mounted on the respective terminal bases 42a and 44a, which are electrically connected respectively to the first electrode 38 and the second electrode 40, through predetermined conductive members (sliver paste, for example). The connection terminals 50, 52 include respective projections 50a, 52a, each of which has a small-diameter cylinder and projects upward. The other portions of the connection terminals 50, 52 than the projections 50a, 52a, together with the terminal bases 42a, 44a, and the connection lines 42b, 44b, are covered with an insulating film (a hook cover) 54. The insulating film 54 has a pair of holes formed therein through which the projections 50a, 52a penetrate. Accordingly, only the projections 50a, 52a are exposed from the insulating film 54, and the surfaces of the other members of the reference-side region 24 are covered with the insulating film 54.

As shown in FIG. 1, the energization device 14 has connection holes 14a and 14b formed on the bottom surface thereof. The projections 50a, 52a of the connection terminals 50, 52 are connected to the respective connection holes 14a, 14b. The energization device 14 has a battery 56 and a current control unit (not shown) housed therein. The electrical configuration of the transdermal drug administration device 10 will be described in detail later.

In the energization device 14, an insulating sheet 58 is interposed between the anode of the battery 56 and the current control unit. Prior to the use of the device 10, the insulating sheet 58 prevents direct current from flowing from the battery 56 to the current control unit. On the other hand, at the start of using the device 10, the insulating sheet 58 is pulled away to electrically connect the anode of the battery 56 and the electrode film 20 to each other. As a result, direct current is supplied from the battery 56 to the donor gel 30, the body of the patient, and the reference gel 34 thorough the electrode film 20. A power supply switch or the like may, of course, be provided instead of the insulating sheet 58.

The device 10 configured as above provides a current path along which the energization device 14 can supply direct current from the connection terminal 50 connected to the connection hole 14a through the terminal base 42a, the connection line 42b, the connection line 38a, and the first electrode 38 to the donor gel 30, and the direct current supplied to the donor gel 30 flows through the body of the patient, the reference gel 34, the second electrode 40, the connection line 40a, the connection line 44b, the terminal base 44a, and the connection terminal 52, and then returns to the energization device 14 through the connection hole 14b.

In this case, in the patch 12 according to the present embodiment, the contact terminal lines 42 and 44, and the connection terminals 50 and 52 for connecting and placing the energization device 14 are provided only on the surface of the reference portion 18.

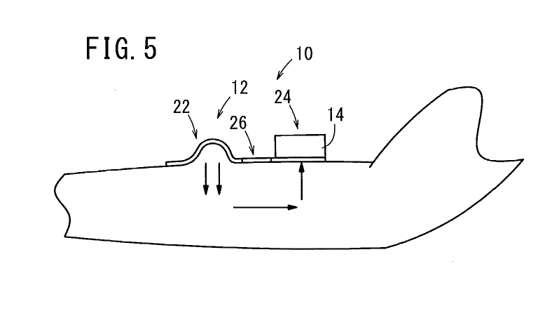

Therefore, of the pair of gels serving as a pair of electrodes to be applied to the skin of the patient, the donor portion 16 which contains the donor gel 30 holding a medical agent is free of the energization device 14. Therefore, the flexibility of the donor portion 16 is not impaired by the energization device 14. Therefore, as shown in FIG. 5, the donor portion 16 containing the medical agent can be easily fixed in intimate contact even with a highly raised blood vessel in a puncture portion (a shunt portion formed on an arm) of a hemodialysis patient with the donor portion 16 closely conforming thereto. In this case, since the donor portion 16 and the reference portion 18 are arranged so as to be distanced from each other with the bridge region 26 interposed therebetween, it is possible to further improve the flexibility of the donor portion 16 and increase the degree of freedom of application. In addition, since the energization device 14 can be directly connected to the patch 12, there is also an advantage that the overall size of the device 10 is reduced, and the device 10 thereby becomes easy to handle.

The energization device 14 has an LED 60a which indicates that the energization is being performed normally, and an LED 60b which alerts that a part of the patch 12 comes off the skin and a contact area S of the donor gel 30 and the reference gel 34 with the skin thereby becomes smaller than a predetermined area. The predetermined area is the minimum area required for a medical professional to perform the puncture therein.

Next, the electrical structure of the transdermal drug administration device 10 will be described in detail. The transdermal drug administration device 10 of the present embodiment changes direct electric current to be supplied depending on the contact area S between the patch 12 (more specifically, the donor gel 30 and the reference gel 34) and the skin.

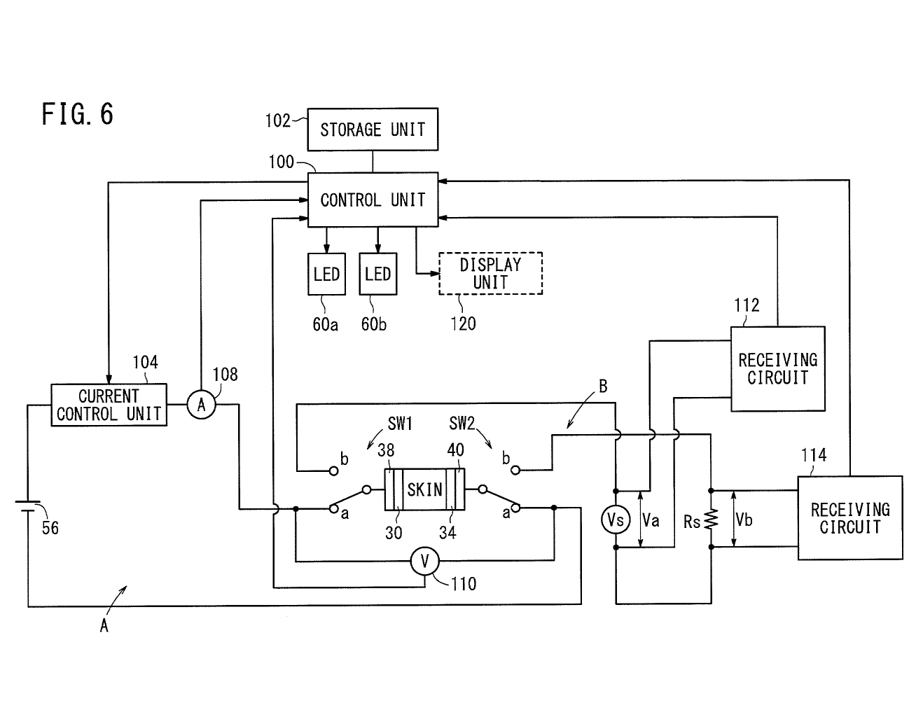

FIG. 6 is a circuit diagram of the transdermal drug administration device 10 in a state where the patch 12 of the transdermal drug administration device 10 is applied to a skin. The “skin” in FIG. 6 indicates an impedance Z between the donor gel 30 and the reference gel 34 (i.e., between the first electrode 38 and the second electrode 40) when the patch 12 is applied to the skin. That is, the impedance Z is a ratio between voltage and current of an external conductor in a path between the donor gel 30 and the reference gel 34 in which current flows. The impedance Z is a resistance value when direct current is supplied. In the case of iontophoresis, the impedance Z of the external conductor is generally considered as a parallel circuit of a resistance R and a capacitance C. Therefore, the impedance Z can be represented by Formula 1 shown below. In the present embodiment, when the impedance Z is calculated, the resistances of the donor gel 30, the reference gel 34, the first electrode 38, and the second electrode 40 are ignored for convenience sake.

$$Z = \frac{R}{1 + jω RC} \\ Therefore , \\ \vert Z \vert = \frac{1}{\sqrt{{( \frac{1}{R} )}^{2} + {( ω C )}^{2}}} \qquad [ Formula 1 ]$$

ω: Angular frequency

The transdermal drug administration device 10 has the battery 56, a first circuit (hereinafter, referred to as a circuit A) for administering a medical agent to a skin of a patient, a second circuit (hereinafter, referred to as a circuit B) for obtaining the contact area S between the patch 12 and the skin, a control unit 100 which has a clock circuit and also serves as a timer, a storage unit 102 in which an energization current table is stored, a current control unit 104 for controlling direct current flowing from the battery 56 and between the first electrode 38 and the second electrode 40 (flowing through the external conductor), the LED 60a which emits green light, and the LED 60b which emits yellow right.

In the circuit A, there are provided a current detection unit 108 which detects direct current flowing between the donor gel 30 and the reference gel 34 (between the first electrode 38 and the second electrode 40), and a voltage detection unit 110 which detects a voltage between the donor gel 30 and the reference gel 34 (between the first electrode 38 and the second electrode 40), that is, a voltage across the impedance Z.

The circuit B has an alternating current power supply Vs having an output impedance inside thereof, and a current detection resistance Rs. The first electrode 38 and the donor gel 30 are connected to the second electrode 40 and the reference gel 34 through the alternating current power supply Vs and the current detection resistance Rs which are connected to each other in series. An electric current flows from the alternating current power supply Vs to the donor gel 30, and is detected by the current detection resistance Rs. The alternating current power supply Vs may output a rectangular wave, and may include, for example, a direct current power supply such as a battery and a switch for switching on/off which are connected to each other in series. A rectangular wave can be output by the on/off operation of the switch.

In the circuit B, there are provided a receiving circuit 112 which detects a voltage Va across the alternating current power supply Vs, and a receiving circuit 114 which detects a voltage Vb across the current detection resistance Rs. When taking the output impedance of the alternating current power supply into consideration, the original voltage of the alternating current power supply Vs, i.e., a no-load voltage thereof, does not match the voltage Va.

The connection state of the first electrode 38 and the second electrode 40 (the donor gel 30 and the reference gel 34) can be switched, by a switch SW1 and a switch SW2, between connection to the circuit A and connection to the circuit B. When the switch SW1 and the switch SW2 are connected to respective terminals a, the first electrode 38 and the second electrode 40 are connected to the circuit A. When the switch SW1 and the switch SW2 are connected to respective terminals b, the first electrode 38 and the second electrode 40 are connected to the circuit B. The switching operation of the switch SW1 and the switch SW2 is controlled by the control unit 100.

Further, when the first electrode 38 and the second electrode 40 are connected to the circuit A, the control unit 100 calculates the resistance R between the first electrode 38 and the second electrode 40 (between the donor gel 30 and the reference gel 34) on the basis of a current I detected by the current detection unit 108 and a voltage V detected by the voltage detection unit 110. More specifically, the resistance R is calculated according to Formula 2 shown below.

$$R = \frac{V}{I} \qquad [ Formula 2 ]$$

Further, when the first electrode 38 and the second electrode 40 are connected to the circuit B, the control unit 100 calculates the impedance Z between the first electrode 38 and the second electrode 40 on the basis of the voltage Va across the alternating current power supply Vs detected by the receiving circuit 112 and the voltage Vb across the current detection resistance Rs detected by the receiving circuit 114. More specifically, the impedance Z is calculated according to Formula 3 shown below.

Z=(Va−Vb)/(Vb/Rs) [Formula 3]

The control unit 100 obtains the capacitance C between the first electrode 38 and the second electrode 40 on the basis of Formulae 1 to 3. The capacitance C decreases in proportion to the contact area S of the donor gel 30 and the reference gel 34. Therefore, the decrease of the capacitance C indicates that the contact area S also decreases in proportion thereto.

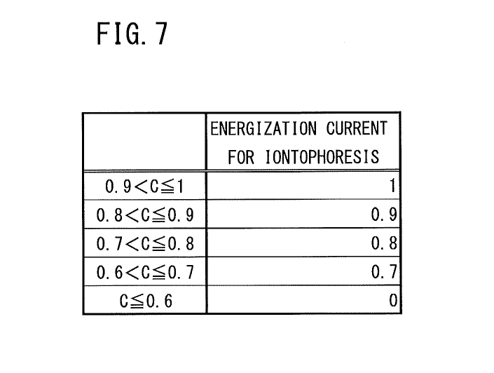

FIG. 7 is a diagram illustrating the energization current table stored in the storage unit 102. Direct current (energization current) to be supplied depending on the capacitance C is stored in the energization current table. A value of the capacitance C that is first obtained after applying the patch 12 to the skin is represented as 1. A value of the energization current (initial current) when the capacitance C is 1 is represented as 1. This is because, immediately after applying the patch 12 to the skin, the contact surfaces of the donor gel 30 and the reference gel 34 can be considered to be in complete contact with the skin. Therefore, the capacitance C at this point is defined as 1. The area of the contact surfaces at this point is defined as an initial area. Further, the initial current has a value at which the density of current flowing into the skin becomes at least equal to or lower than a threshold value when the contact surfaces of the donor gel 30 and the reference gel 34 are in complete contact with the skin. When the current density is larger than the threshold value, the patient feels pain, and the skin is also adversely affected thereby. In the present embodiment, direct current is supplied to the skin such that the current density falls within a certain range from a predetermined value. It is needless to say that the current density that falls within the certain range from the predetermined value is equal to or lower than the threshold value. Strictly describing, the current density of the energization current is a value calculated by dividing the energization current by the contact area S.

As shown in FIG. 7, when the capacitance C satisfies 0.9<C≦1 (the initial area), the energization current becomes 1 (the initial current). That is, when the contact area S is larger than 90% of the initial area, the initial current is supplied. When the capacitance C satisfies 0.8<C≦0.9, the energization current becomes 0.9. That is, when the contact area S is larger than 80%, but equal to or less than 90% of the initial area, a current that is 90% of the initial current is supplied.

When the capacitance C satisfies 0.7<C≦0.8, the energization current becomes 0.8. That is, when the contact area S is larger than 70%, but equal to or less than 80% of the initial area, a current that is 80% of the initial current is supplied. When the capacitance C satisfies 0.6<C≦0.7, the energization current becomes 0.7. That is, when the contact area S is larger than 60%, but equal to or less than 70% of the initial area, a current that is 70% of the initial current is supplied. When the capacitance C satisfies C≦0.6, the energization current becomes 0. That is, when the contact area S is equal to or less than 60% of the initial area, a current to be supplied is 0.

The current control unit 104 controls direct current flowing between the first electrode 38 and the second electrode 40 to be basically constant under the control of the control unit 100. The current control unit 104, for example, has a boosted switching power supply, and is capable of controlling the direct current flowing between the first electrode 38 and the second electrode 40 to be constant by changing a period during which the switching is performed to thereby change the direct current voltage, in accordance with the control by the control unit 100 to which a current detected by a current detection unit described below is input. Further, the current control unit 104, for example, has a switch for connecting and disconnecting the circuit A, and is capable of controlling start and stop of supply of current between the first electrode 38 and the second electrode 40 by switching on/off of the switch in accordance with the control by the control unit 100. Further, the current control unit 104 may control start and stop of supply of current between the first electrode 38 and the second electrode 40 by controlling the switch SW1 and the switch SW2. In particular, the current control unit 104, for example, may connect the switch SW1 to the terminal a and connects the switch SW2 to the terminal b.

The LED 60a emits green light under the control of the control unit 100. On starting energization to the first electrode 38, the control unit 100 drives the LED 60a so as to emit green light. When the control unit 100 determines that the capacitance C has come to C≦0.6, the control unit 100 drives the LED 60b so as to emit yellow light. Further, when an administration time for administering the medical agent is finished, the energization is finished. Therefore, the control unit 100 finishes the light emission of the LED 60a.

The control unit 100, the storage unit 102, the current control unit 104, the current detection unit 108, the voltage detection unit 110, the receiving circuit 112, the receiving circuit 114, the switch SW1, and the switch SW2 are provided in the energization device 14. The first electrode 38, the second electrode 40, the donor gel 30, and the reference gel 34 are provided in the patch 12 as described above. Further, the control unit 100 includes a computer. The computer reads a predetermined program stored in the storage unit 102, and thereby serves as the control unit 100.

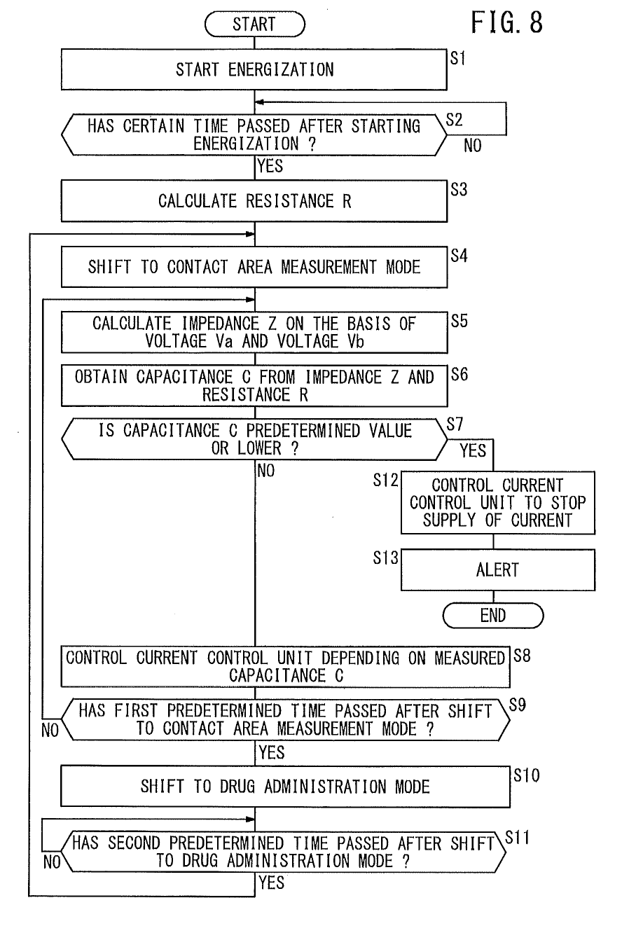

Next, the operation of the control unit 100 will be described according to the flowchart of FIG. 8.

By applying the transdermal drug administration device 10 to a skin of a patient, and then pulling away the insulating sheet 58, a direct current is supplied from the current control unit 104 to the skin (external conductor) through the donor gel 30, and energization is thereby started (step S1). At this time, the control unit 100 drives the LED 60a so as to emit green light. As shown in FIG. 6, the switches SW1 and SW2 are connected to the respective terminals a by the control unit 100 in step S1. The current control unit 104 is controlled by the control unit 100 such that the initial current can be supplied to the donor gel 30 when the impedance Z of the skin is stabilized as described later. For example, the output current of the current control unit 104 is set to an initial current value by the control unit 100. More specifically, the value of the current flowing between the first electrode 38 and the second electrode 40 when the impedance Z is stabilized as described later is the initial current value.

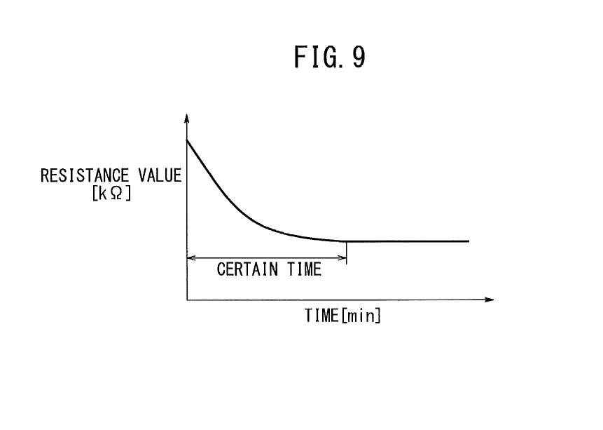

Next, a determination is made as to whether a certain time (at least a period of time for stabilizing the impedance Z) has passed after starting the energization (step S2). As shown in FIG. 9, immediately after starting the supply of direct current to the donor gel 30, the drug has not yet infiltrated the skin or the human body, and the impedance Z is therefore high. Thereafter, the drug infiltrates the skin or the like, and the impedance Z thereby gradually decreases with the lapse of time. Eventually, the impedance Z becomes substantially constant. On the condition that the contact area S is constant, the energization current gradually increases while the impedance Z gradually decreases, and when the impedance Z becomes substantially constant, the energization current also becomes the initial current (substantially constant).

In step S2, when it is determined that the certain period of time has not yet passed after starting the energization, the process remains in step S2 until the certain period of time passes. When it is determined that the certain period of time has passed after starting the energization, the resistance R between the first electrode 38 and the second electrode 40 is calculated (step S3). More specifically, the resistance R between the first electrode 38 and the second electrode 40 is calculated on the basis of the current I flowing between the first electrode 38 and the second electrode 40 detected by the current detection unit 108 and the voltage V (the voltage across the impedance Z) between the first electrode 38 and the second electrode 40 detected by the voltage detection unit 110. More specifically, the resistance R can be calculated by Formula 2 described above.

Next, the process goes to a contact area measurement mode (step S4). When the process goes to the contact area measurement mode, the control unit 100 connects the switches SW1 and SW2 to the respective terminals b. Accordingly, an alternating current is supplied between the first electrode 38 and the second electrode 40 by the alternating current power supply Vs. As this time, the alternating current is supplied to the skin at a current density which is at least equal to or lower than the threshold value. Since the alternating current is supplied to the skin in order to obtain the impedance Z, the alternating current may be considerably lower than the direct current supplied from the battery 56. As a result, even when a part of the patch 12 comes off the skin, and the contact area S thereby decreases, the current density of the alternating current is lower than the threshold value.

When the process is shifted to the contact area measurement mode, the impedance Z is calculated on the basis of the voltage Va detected by the receiving circuit 112 and the voltage Vb detected by the receiving circuit 114 (step S5). The impedance Z can be calculated according to Formula 3 described above.

Next, the capacitance C is calculated from the resistance R calculated in step S3 and the impedance Z calculated in step S5 (step S6). The capacitance C can be calculated by Formula 1 described above. The capacitance C represents the contact area S of the donor gel 30 and the reference gel 34 with the skin. According to Formula 1, the capacitance C can be calculated only from the resistance R and the impedance Z calculated with a certain angular frequency. However, in order to obtain a more accurate value, the capacitance C may be calculated on the basis of Formula 1 while varying the angular frequency.

Next, a determination is made as to whether the capacitance C calculated in step S6 is a predetermined value (step S7). The determination is made by using the energization current table stored in the storage unit 102. That is, a determination is made as to whether the capacitance C satisfies C≦0.6. Here, the value of the capacitance C first obtained in step S6 is defined as 1. Therefore, the predetermined value is 60% of the first obtained capacitance C. This is because, when the capacitance C is equal to or less than 60%, the contact area S becomes smaller than the predetermined area that is the minimum area required for a medical professional to perform puncture. Examples of a factor of the detachment of the patch 12 include a physical exercise of the patient (a movement for moving the arm) and sweating. The contact area S may decrease with the lapse of time due to these factors.

In step S7, when it is determined that the capacitance C is not equal to or lower than the predetermined value, the current control unit 104 is controlled so as to supply a direct current depending on the measured capacitance C between the first electrode 38 and the second electrode 40 (step S8). The control is performed by using the energization current table stored in the storage unit 102. For example, when the capacitance C satisfies 0.9<C≦1, a direct current of 1 (the initial current) is continuously supplied between the first electrode 38 and the second electrode 40. When the capacitance C satisfies 0.8<C≦0.9, the current control unit 104 is controlled so as to supply a direct current of 0.9 (a direct current that is 90% of the initial current) between the first electrode 38 and the second electrode 40. When the capacitance C satisfies 0.7<C≦0.8, the current control unit 104 is controlled so as to supply a direct current of 0.8 (a direct current that is 80% of the initial current) between the first electrode 38 and the second electrode 40. When the capacitance C satisfies 0.6<C≦0.7, the current control unit 104 is controlled so as to supply a direct current of 0.7 (a direct current that is 70% of the initial current) between the first electrode 38 and the second electrode 40. Accordingly, even when the contact area S decreases, the current density of the energization current flowing in the skin falls within the certain range from the predetermined value (the current density of energization current can be kept substantially constant), thereby making it possible to continue the energization without causing an adverse effect on the skin.

Next, a determination is made as to whether a first predetermined time (one second, for example) has passed after shifting the process to the contact area measurement mode (step S9). When it is determined that the first predetermined time has not yet passed, the process returns to step S5, and the above operations are repeated. A period during which the contact area measurement mode is performed is referred to as a first period.

On the other hand, when it is determined that the first predetermined time has passed after shifting the process to the contact area measurement mode in step S9, the process goes to a drug administration mode (step S10). When the process goes to the drug administration mode, the control unit 100 connects the switches SW1 and SW2 to the respective terminals a. Accordingly, a direct current is supplied between the first electrode 38 and the second electrode 40. At this time, the current density of the direct current flowing in the skin is maintained within the certain range from the predetermined value by the control in step S8.

Next, a determination is made as to whether a second predetermined time (nine seconds, for example) has passed after shifting the process to the drug administration mode (step S11). When it is determined that the second predetermined time (a second period) has not yet passed, the process remains in step S11 until the second predetermined time passes. On the other hand, when it is determined that the second predetermined time has passed after shifting the process to the drug administration mode in step S11, the process returns to step S4, and the above operations are repeated. A period during which the drug administration mode is performed is referred to as the second period. During the second period, a direct current is supplied between the first electrode 38 and the second electrode 40 at a predetermined cycle (ten seconds, for example).

On the other hand, when a part of the patch 12 comes off the skin with the lapse of time, and it is therefore determined that the obtained capacitance C is equal to or lower than the predetermined value (i.e., when it is determined that the calculated capacitance C satisfies C≦0.6, where the first obtained capacitance C is defined as 1) in step S7, the current control unit 104 is controlled to stop the supply of current (step S12). That is, the control unit 100 connects the switches SW1 and SW2 to the respective terminals a, and controls the current control unit 104 to stop the supply of direct current. This is because, when the drug is lidocaine and the lidocaine is administered by the transdermal drug administration device 10, it is not possible to administer the lidocaine because the contact area S is smaller than the minimum area required for a medical professional to perform puncture.

Next, the control unit 100 alerts the patient or the medical professional to the fact that the contact area S is smaller than the predetermined area (step S13), and then finishes the process. More specifically, the control unit 100 drives the LED 60b so as to emit yellow light, and stops the drive of the LED 60a so as to stop the emission of green light.

Also when it is determined that the obtained capacitance C is not equal to or lower than the predetermined value in step S7, and a time required for the drug administration (an administration time) has passed, the control unit 100 controls the current control unit 104 to stop the supply of direct current. At this time, the control unit 100 stops the drive of the LED 60a to thereby stop the emission of the green light. A determination as to whether the administration time has passed may be made on the basis of a starting time of the energization, or may also be made on the basis of a point at which a certain period of time has passed after starting the energization.

As described above, a direct current is supplied between the donor gel 30 and the reference gel 34 (i.e., supplied to the skin) at a predetermined cycle, and an alternating current is supplied during the first period in which a direct current is not supplied, whereby the capacitance C is obtained. Thus, it is possible to indirectly know the contact area S of the donor gel 30 and the reference gel 34 with the skin, the contact area S varying in proportion to the capacitance C. This makes it possible to supply a direct current such that the density of the current flowing in the skin of a patient is kept substantially constant. As a result, even when the contact area S decreases, it is possible to continue the energization without causing an adverse effect on the skin of the patient.

Since the impedance Z is obtained during the first period, and the resistance R is obtained during the second period in which a direct current is supplied, it is possible to accurately obtain the capacitance C, and therefore possible to accurately obtain the contact area S.

Since the switches SW1 and SW2 are connected to the respective terminals b during the first period, and connected to the respective terminals a during the second period, it is possible to selectively supply a direct current and an alternating current between the first electrode 38 and the second electrode 40.

Since the value of direct current to be supplied to the first electrode 38 is controlled depending on the obtained capacitance C, the density of the direct current flowing in the skin of a patient can be kept constant. Therefore, even when a part of the patch 12 comes off the skin with the lapse of time, it is possible to continue the energization without causing an adverse effect on the skin of the patient.

When the medical agent is an anesthetic such as lidocaine, and the obtained capacitance C is equal to or lower than the predetermined value (i.e., when the contact area S becomes smaller than the minimum predetermined area required for puncture), the supply of direct current to the first electrode 38 is stopped. Therefore, it is possible to prevent an adverse effect on the skin of the patient due to excessive current density. In addition, it is possible to prevent the medical agent from being wastefully administered.

Further, when the medical agent is an anesthetic such as lidocaine, and the obtained capacitance C is equal to or lower than the predetermined value (i.e., when the contact area S becomes smaller than the minimum predetermined area required for puncture), an alert is issued. Therefore, a patient or a medical professional can recognize that a part of the patch 12 has come off the skin, and the contact area S has thereby become smaller than the predetermined area.

Although the administration of an anesthetic such as lidocaine has been described in the above embodiment, a medical agent other than an anesthetic may be administered.

[Modifications] The above embodiment may be modified in the following manner.

(Modification 1) In the above embodiment, an alert is issued to a patient or a medical professional by driving the LED 60b so as to emit yellow light in step S13 of FIG. 8. However, the energization device 14 may be provided with a display unit 120 (see FIG. 6), and the control unit 100 may display the fact that the patch 12 has come off the skin on the display unit 120, thereby issuing an alert. Further, the energization device 14 may be provided with a speaker, and the control unit 100 may issue an alert by, for example, emitting an alert sound from the speaker.

(Modification 2) In the above embodiment and Modification 1 described above, the supply of current is stopped when the capacitance C is equal to or less than the predetermined value in step S12 of FIG. 8. However, the energization may be continued without stopping the supply of current. This is because, in this case, a user can re-apply the patch 12 by virtue of the alert in step S13. Further, when a medical agent other than an anesthetic for puncture is administered, even if the contact area S is small, there is no effect caused. Therefore, even when the contact area S is equal to or less than the predetermined value, the energization may be continued. Also in this case, it is needless to say that the energization current should be controlled depending on the capacitance C.

(Modification 3) In the above embodiment and Modifications 1 and 2 described above, the density of the direct current flowing in the skin is made to fall within a certain range from a predetermined value by supplying the direct current depending on the capacitance C between the first electrode 38 and the second electrode 40 in step S8 of FIG. 8. However, the density of the direct current flowing in the skin does not have to be within the certain range from the predetermined value as long as the current density is equal to or less than a threshold value.

(Modification 4) In the above embodiment and Modifications 1 to 3 described above, the energization device 14 may be provided with the display unit 120 (see FIG. 6), and the control unit 100 may display a remaining energization time on the display unit 120. That is, a time to complete the administration of the medical agent may be displayed thereon. Further, in the above embodiment and Modifications 1 and 2 described above, even when a part of the patch 12 comes off the skin, and the contact area S of the donor gel 30 and the reference gel 34 with the skin thereby decreases, the energization is stopped when the administration time of the medical agent (10 minutes, for example) has passed. However, when the contact area S decreases, the energization current also decreases, and the medical agent administered to the skin therefore also decreases along with the decrease of the energization current. Therefore, in view of the contact area S, the administration time of the medical agent is also changed, and a remaining energization time with respect to the changed administration time may be displayed.

In this regard, when the patch 12 does not come off, and the contact surfaces of the donor gel 30 and the reference gel 34 are in complete contact with the skin (i.e., when the contact area S is the initial area), the administration time of the medical agent is defined as an initial administration time (10 minutes, for example). In a case where the contact area S of the donor gel 30 and the reference gel 34 with the skin is the initial area, it is possible to administer a certain amount of medical agent to the skin by performing the energization only for the initial administration time. Therefore, in this case, a time calculated by subtracting a time for which the energization has been performed, from the initial administration time is the remaining energization time. When the remaining energization time becomes zero, the energization should be stopped.

However, when a part of the patch 12 comes off the skin, and the contact area S of the donor gel 30 and the reference gel 34 with the skin thereby decreases, the direct current flowing in the skin also decreases. Therefore, in order to administer the certain amount of medical agent, it is necessary to perform the energization for a longer period of time than the initial administration time, and the administration time is therefore required to be changed. Hereinafter, an example of a method for changing the administration time will be described in detail. Here, a description will be given by comparing a case where energization is performed with the contact area S of the donor gel 30 and the reference gel 34 with the skin remaining at the initial area with a case where energization is performed with the contact area S remaining smaller than the initial area from the start.

The amount of the drug to be administered is represented by the energization time and the energization current. In the case where energization is performed with the contact area S of the donor gel 30 and the reference gel 34 with the skin remaining at the initial area, the certain amount of a drug to be administered (hereinafter, referred to as a certain amount M) is represented by the initial administration time (hereinafter, referred to as an initial administration time T1)×the initial current (hereinafter, referred to as an initial current I1).

On the other hand, in the case where energization is performed with the contact area S remaining smaller than the initial area from the start, the amount of a drug to be administered (hereinafter, referred to as an amount m) is represented by the administration time (hereinafter, referred to as an administration time T2)×the energization current corresponding to the contact area S (hereinafter, referred to as an energization current I2).

The certain amount M is equal to the amount m. Therefore, a time T for which the drug is administered when the contact area S decreases can be calculated by a relational expression of the administration time T1×the initial current I1/the energization current I2. That is, a current should be supplied until a charge amount based on the supplied current becomes equal to a value represented by the multiplication of the initial current I1 and the initial administration time T1 (mA·sec). The administration time can be calculated depending on the energization current corresponding to the present contact area S.

(Modification 5) In the above embodiment and Modifications 1 to 4 described above, the descriptions have been given with regard to the transdermal drug administration device 10. However, a transdermal drug administration device 200 described below may be used. In Modification 5, those parts which are identical or similar to those in the above embodiment are denoted by the same reference numerals, and only different parts will be mainly described.

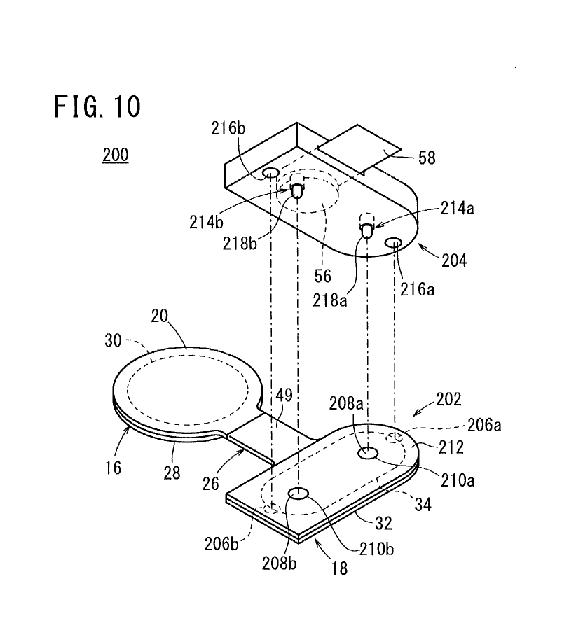

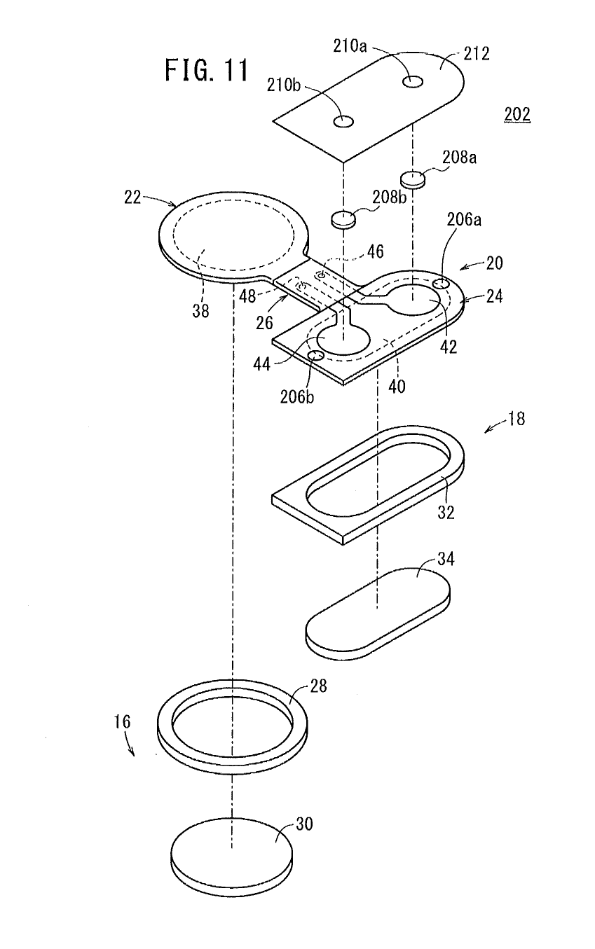

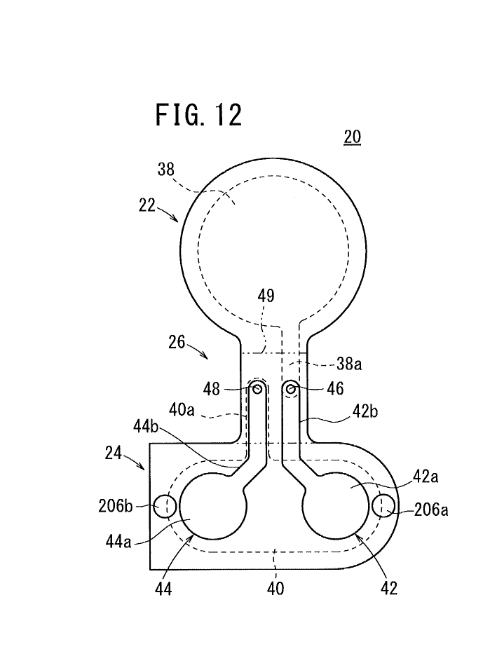

FIG. 10 is a perspective view illustrating the overall structure of the transdermal drug administration device 200. In FIG. 10, an iontophoresis patch 202 and an energization device 204 which constitute the transdermal drug administration device 200 (hereinafter, referred to as the device 200) are illustrated as being separated from each other. FIG. 11 is an exploded perspective view of the iontophoresis patch (hereinafter, referred to as the patch) 202 shown in FIG. 10. FIG. 12 is a top plan view of the electrode film 20.

As shown in FIGS. 10 and 11, the device 200 is provided with the patch 202, and the energization device 204 which is placed on and connected to the surface (the upper surface) of the patch 202.

As shown in FIG. 12, a pair of magnetic bodies 206a, 206b, each of which has a circular shape and includes a thin iron plate, is provided on the upper surface (the surface making contact with the energization device 204) of the electrode film 20. Further, rigid conductive plates 208a and 208b, each of which has a circular shape and includes a thin iron plate, are respectively provided on the upper surfaces of the terminal bases 42a and 44a (see FIG. 12) of the reference-side region 24. The circumferential edges of the conductive plates 208a and 208b, the terminal bases 42a and 44a, and the connection lines 42b and 44b are covered with an insulating film 212 having two holes 210a and 210b. Accordingly, remaining parts of the conductive plates 208a and 208b, the remaining parts being uncovered due to the two holes 210a and 210b, are exposed from the insulating film. Each of the conductive plates 208a and 208b is more rigid than the base 36 (see FIG. 3).

The positions of the centers of the terminal base 42a, the conductive plate 208a, and the hole 210a coincide with each other (that is, a straight line that passes through the center of the terminal base 42a and is perpendicular to a plane of the terminal base 42a passes through the centers of the conductive plate 208a and the hole 210a). Further, the positions of the centers of the terminal base 44a, the conductive plate 208b, and the hole 210b coincide with each other (that is, a straight line that passes through the center of the terminal base 44a and is perpendicular to a plane of the terminal base 44a passes through the centers of the conductive plate 208b and the hole 210b).

As shown in FIG. 10, the energization device 204 has a pair of spring probes (contact terminals for energization) 214a and 214b and a pair of permanent magnets (first magnets) 216a and 216b all of which are disposed on the bottom surface thereof. The spring probes 214a and 214b make electrical contact with the conductive plates 208a and 208b, respectively, which are exposed through the two holes 210a and 210b of the insulating film 212 of the patch 202. The energization device 204 has the battery 56, the control unit 100, etc. housed therein (see FIG. 6). As shown in FIG. 10, the permanent magnets 216a and 216b are disposed on the bottom surface of the energization device 204 so as to attract the magnetic bodies 206a and 206b, respectively (so as to face the magnetic bodies 206a and 206b, respectively) when the energization device 204 is attached to the patch 202 such that the spring probes 214a and 214b make contact with the conductive plates 208a and 208b, respectively, which are exposed from the two holes 210a and 210b. That is, the permanent magnets 216a and 216b serve to position the energization device 204 relative to the patch 202, and also have a function to attach the energization device 204 to the patch 202. An end terminal 218a of the spring probe 214a and an end terminal 218b of the spring probe 214b are biased by springs (not shown) so as to protrude downward from the bottom surface of the energization device 204. The terminal bases 42a and 44a, the conductive plates 208a and 208b, and the spring probes 214a and 214b constitute a main part of the conduction mechanism for iontophoresis.

When the energization device 204 is brought into contact with the patch 202 in the orientation shown in FIG. 10 (in a predetermined orientation), the permanent magnets 216a and 216b of the energization device 204 attract the magnetic bodies 206a and 206b of the patch 202, respectively, thereby making it possible to fix the energization device 204 to the patch 202. At this time, the end terminal 218a of the spring probe 214a and the end terminal 218b of the spring probe 214b press the conductive plates 208a and 208b, respectively, in a direction to separate the energization device 204 and the patch 202 from each other. However, the pressing force is weaker than the magnetic attractive force of the permanent magnets 216a and 216b. Therefore, the energization device 204 and the patch 202 do not come to be separated from each other. Accordingly, the end terminal 218a of the spring probe 214a and the end terminal 218b of the spring probe 214b are in firm contact with the conductive plates 208a and 208b, respectively. As a result, the electrical connection between the energization device 204 and the patch 202 can be made firm. In order to achieve firmer electrical connection between the energization device 204 and the patch 202, the two holes 210a and 210b are preferably provided between the pair of magnetic bodies 206a and 206b. This makes it possible to achieve firmer contact between the end terminal 218a of the spring probe 214a and the conductive plate 208a and between the end terminal 218b of the spring probe 214b and the conductive plate 208b.

As described above, in Modification 5, the energization device 204 has the pair of spring probes 214a and 214b for supplying an electric current to the patch 202. The patch 202 has the pair of terminal bases 42a and 44a which are electrically connected to the pair of spring probes 214a and 214b. By causing the patch 202 and the energization device 204 to attract each other by the magnetic force of the permanent magnets 216a and 216b, the pair of spring probes 214a and 214b is electrically connected to the pair of terminal bases 42a and 44a. Therefore, it is not necessary to provide a connector, thereby making it possible to provide a transdermal drug administration device at a low cost.

Further, the energization device 204 has the pair of permanent magnets 216a and 216b for positioning the energization device 204 relative to the patch 202. The patch 202 has the pair of magnetic bodies 206a and 206b which is attracted by the pair of permanent magnets 216a and 216b. When the pair of permanent magnets 216a and 216b attracts the pair of magnetic bodies 206a and 206b, the pair of spring probes 214a and 214b is electrically connected to the pair of terminal bases 42a and 44a. Therefore, it is possible to electrically connect the energization device 204 with the patch 202 easily.

The terminal bases 42a and 44a which are provided in the patch 202 and electrically connected to the energization device 204 are formed by printing electrically conductive materials on the flexible base 36. Therefore, the cost for manufacturing the transdermal drug administration device can further be reduced.

The conductive plates 208a and 208b which are more rigid than the base 36 and respectively make contact with the spring probes 214a and 214b are mounted on the terminal bases 42a and 44a, respectively, and the pair of spring probes 214a and 214b presses the pair of conductive plates 208a and 208b. Therefore, the contact surfaces of the conductive plates 208a and 208b are not deformed by pressing by the spring probes 214a and 214b. Accordingly, it is possible to improve the contact between the spring probe 214a and the conductive plate 208a and between the spring probe 214b and the conductive plate 208b, and thereby improve the electrical connection between the spring probe 214a and the terminal base 42a and between the spring probe 214b and the terminal base 44a, that is, the electrical connection between the energization device 204 and the patch 202. On the other hand, in a case where the conductive plates 208a and 208b are not provided, the spring probes 214a and 214b directly press the terminal bases 42a and 44a, respectively, which are exposed through the holes 210a and 210b of the insulating film 212. Therefore, the terminal bases 42a and 44a mounted on the flexible base 36 may be deformed by the pressing. As a result, contact failure between the spring probe 214a and the terminal base 42a and between the spring probe 214b and the terminal base 44a may occur, and the electrical connection between the energization device 204 and the patch 202 may thereby be cut off. However, since the conductive plates 208a and 208b are provided in the present embodiment, such failure can be prevented.

(Modification 6) The description has been given with reference to the spring probes 214a and 214b in Modification 5. However, basically, the spring probes 214a and 214b may be any contact terminals for energization (energization contact terminals) that can make contact with the respective conductive plates 208a and 208b to allow an electric current to flow.

(Modification 7) In Modifications 5 and 6 described above, the pair of permanent magnets 216a and 216b is provided in the energization device 204, and the pair of magnetic bodies 206a and 206b is provided in the patch 202. However, the pair of magnetic bodies 206a and 206b may be provided in the energization device 204, and the pair of permanent magnets 216a and 216b may be provided in the patch 202. Further, although the two permanent magnets 216a and 216b and the two magnetic bodies 206a and 206b are used in Modifications 5 and 6, a single permanent magnet and a single magnetic body may be used, or three or more permanent magnets and three or more magnetic bodies may be used. For example, a single permanent magnet may be provided in the energization device 204, and a single magnetic body may be provided in the patch 202.

(Modification 8) In Modifications 5 to 7 described above, the conductive plates 208a and 208b are provided in the patch 202. However, the conductive plates 208a and 208b may not be provided. In this case, the spring probes 214a and 214b press the terminal bases 42a and 44a, respectively, which are exposed through the holes 210a and 210b of the insulating film 212. However, by reducing the pressing force (the force of the springs of the spring probes 214a and 214b) depending on the degree of rigidity of the base 36, it is possible to prevent occurrence of contact failure between the spring probe 214a and the terminal bases 42a and between the spring probe 214b and the terminal bases 44a.

(Modification 9)

In Modifications 5 to 8 described above, the holes 210a and 210b are disposed on the insulating film 212 so as to be symmetric with respect to a straight line that is perpendicular to a straight line connecting the centers of the circular magnetic bodies 206a and 206b and passes through the midpoint M of the straight line connecting the centers. Therefore, if the energization device 204 is attached to the patch 202 in an orientation that is opposite to the predetermined orientation (by rotating the energization device 204 by 180 degrees), that is, if the energization device 204 is attached to the patch 202 such that the permanent magnet 216a attracts the magnetic body 206b, and the permanent magnet 216b attracts the magnetic body 206a, the spring probe 214a makes contact with the conductive plate 208b which is exposed through the hole 210b, and the spring probe 214b makes contact with the conductive plate 208a which is exposed through the hole 210a. Therefore, an electric current is supplied from the energization device 204 in an opposite direction. In this Modification 9, such misconnection between the energization device 204 and the patch 202 is prevented.

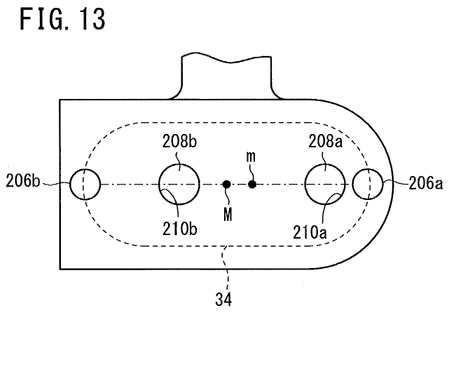

FIG. 13 is a diagram illustrating an example of an arrangement relationship between the holes 210a and 210b and the magnetic bodies 206a and 206b in Modification 9. In FIG. 13, the holes 210a and 210b are disposed on the insulating film 212 so as to be asymmetric with respect to the straight line that is perpendicular to the straight line connecting the centers of the magnetic bodies 206a and 206b and passes through the midpoint M of the straight line connecting the centers. Accordingly, if the energization device 204 is attached to the patch 202 in the orientation that is opposite to the predetermined orientation, the spring probes 214a and 214b do not make contact with the conductive plates 208a and 208b which are respectively exposed through the holes 210a and 210b. Therefore, it is possible to prevent misconnection between the energization device 204 and the patch 202. More specifically, the holes 210a and 210b may be disposed on the insulating film 212 such that the midpoint m of a straight line that connects the centers of the holes 210a and 210b and the midpoint M of the straight line that connects the centers of the magnetic bodies 206a and 206b do not coincide with each other.

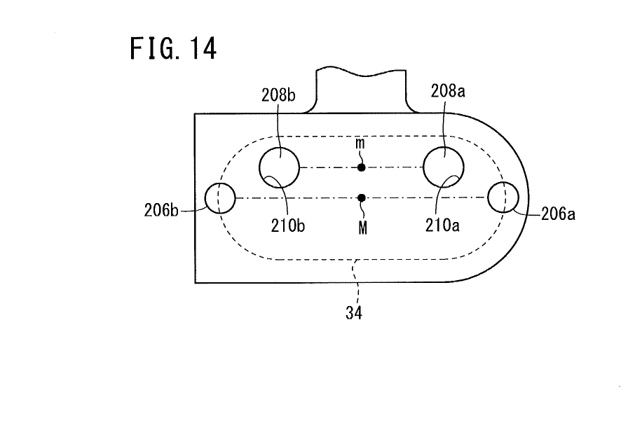

FIG. 14 is a diagram illustrating another example of the arrangement relationship between the holes 210a and 210b and the magnetic bodies 206a and 206b in this Modification 9. In FIG. 14, the holes 210a and 210b are disposed on the insulating film 212 so as to be symmetric with respect to the straight line that is perpendicular to the straight line connecting the centers of the magnetic bodies 206a and 206b and passes through the midpoint M of the straight line connecting the centers, but such that the midpoint m of the straight line connecting the centers of the holes 210a and 210b does not coincide with the midpoint M. Accordingly, when the energization device 204 is attached to the patch 202 in the orientation that is opposite to the predetermined orientation, the spring probes 214a and 214b do not make contact with the conductive plates 208a and 208b which are respectively exposed through the holes 210a and 210b. Therefore, it is possible to prevent misconnection between the energization device 204 and the patch 202.

In this manner, the pair of holes 210a and 210b is disposed on the patch 202 such that the midpoint m of the straight line connecting the centers of the pair of holes 210a and 210b and the midpoint M of the straight line connecting the centers of the pair of magnetic bodies 206a and 206b do not coincide with each other. Therefore, even if a user or the like attempts to attach, or when a user or the like has attached the energization device 204 to the patch 202 in an orientation other than the predetermined orientation, the pair of spring probes 214a and 214b does not make contact with the pair of conductive plates 208a and 208b. Therefore, it is possible to prevent misconnection between the energization device 204 and the patch 202. Here, a radius R of each of the holes 210a and 210b, and a distance L between the midpoint m and the midpoint M are, at least, required to satisfy a relationship of R<2×L. This is because, for example, in a case where R≧2×2L, if the energization device 204 is attached to the patch 202 in the orientation that is opposite to the predetermined orientation, the spring probes 214a and 214b make contact with the conductive plates 208a and 208b which are respectively exposed through the holes 210a and 210b.

Further, it is needless to say that, in FIGS. 13 and 14, the pair of permanent magnets 216a and 216b of the energization device 204 is arranged at such positions that the permanent magnets 216a and 216b attract the respective magnetic bodies 206a and 206b when the energization device 204 is attached to the patch 202 in the predetermined orientation, and the spring probes 214a and 214b of the energization device 204 are arranged at such positions that the spring probes 214a and 214b make contact with the respective conductive plates 208a and 208b, which are respectively exposed through the holes 210a and 210b, when the energization device 204 is attached to the patch 202 in the predetermined orientation.

(Modification 10)

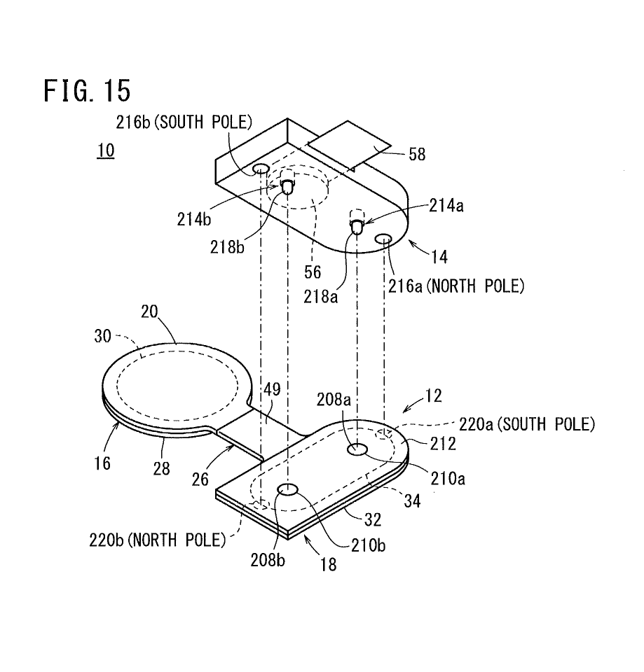

The pair of magnetic bodies 206a and 206b is used in Modifications 5 to 9 described above. However, as shown in FIG. 15, a pair of permanent magnets (second magnets) 220a and 220b may be used instead of the pair of magnetic bodies 206a and 206b. In this case, the pair of permanent magnets 216a and 216b of the energization device 204 is arranged such that magnetic poles of the permanent magnets 216a and 216b at a side that makes contact with the patch 202 are different from each other. Further, the permanent magnet 220a is provided instead of the magnetic body 206a, and the permanent magnet 220b is provided instead of the magnetic body 206b. When the energization device 204 is attached to the patch 202, the permanent magnets 220a and 220b are required to attract the permanent magnets 216a and 216b, respectively. Therefore, the pair of permanent magnets 220a and 220b is arranged in the patch 202 such that a magnetic pole of the permanent magnet 220a at a side that makes contact with the energization device 204 is opposite to a magnetic pole of the corresponding permanent magnet 216a at the side making contact with the patch 202, and a magnetic pole of the permanent magnet 220b at the side making contact with the energization device 204 is opposite to a magnetic pole of the corresponding permanent magnet 216b at the side making contact with the patch 202.

In FIG. 15, the permanent magnets 216a and 216b are arranged in the energization device 204 such that the magnetic poles of the permanent magnets 216a and 216b at the side making contact with the patch 202 are the north pole and the south pole, respectively. The permanent magnets 220a and 220b are arranged in the patch 202 such that the magnetic poles of the permanent magnets 220a and 220b at the side making contact with the energization device 204 are the south pole and the north pole, respectively.

Accordingly, even if a user or the like attempts to attach the energization device 204 to the patch 202 in the orientation that is opposite to the predetermined orientation, the pair of permanent magnets 216a and 216b and the pair of permanent magnets 220a and 220b repel each other. Therefore, the energization device 204 cannot be attached to the patch 202. Thus, it is possible to prevent misconnection between the energization device 204 and the patch 202.

(Modification 11)

In Modifications 5 to 10, as shown in FIGS. 10 and 11, the conductive plates 208a and 208b are respectively exposed through the holes 210a and 210b of the insulating film 212. However, the conductive plates 208a and 208b may be disposed over the holes 210a and 210b of the insulating film 212, thereby allowing the conductive plates 208a and 208b to make contact with the spring probes 214a and 214b, respectively.

Further, in the above embodiment, the insulating film 212 is disposed on the reference-side region 24 as shown in FIGS. 10 and 11. However, the insulating film 212 may not be provided. In this case, the diameter of each of the terminal bases 42a and 44a may be made smaller so as to be equal to the diameter of each of the holes 210a and 210b, and the terminal bases 42a and 44a may be arranged at the same positions as the respective holes 210a and 210b. Further, the insulating film 212 that covers only the connection lines 42b and 44b may be disposed on the reference-side region 24.

Although the present invention has been described with reference to the preferred embodiment thereof, the technical scope of the present invention is not limited to the range described in the aforementioned embodiment. Various alterations or modifications of the above embodiment will become apparent to those skilled in the art. It will be apparent from the description of the appended claims that altered or modified embodiments can also fall within the technical scope of the present invention.

1. A transdermal drug administration device comprising:
a patch provided with a donor portion having a first contact member containing therein a medical agent, a reference portion having a second contact member, a first electrode connected to the first contact member, and a second electrode connected to the second contact member; and
a control unit for controlling supply of an electric current to the first electrode,
wherein, by applying the patch to an external conductor, the first contact member and the second contact member come into contact with the external conductor, and the first electrode and the second electrode are thereby electrically connected to the external conductor, and
when the patch is applied to the external conductor, the control unit supplies a direct current between the first electrode and the second electrode at a predetermined cycle, and supplies an alternating current between the first electrode and the second electrode during a first period in which a direct current is not supplied, thereby obtaining a capacitance between the first electrode and the second electrode.

2. The transdermal drug administration device according to claim 1, wherein the control unit obtains an impedance between the first electrode and the second electrode during the first period, obtains a resistance between the first electrode and the second electrode during a second period in which a direct current is supplied, and obtains the capacitance from the impedance and the resistance.

3. The transdermal drug administration device according to claim 1, further comprising:
a first circuit for supplying a direct current between the first electrode and the second electrode;
a second circuit for supplying an alternating current between the first electrode and the second electrode; and
a switch for switching connection of the first electrode and the second electrode between connection to the first circuit and connection to the second circuit,
wherein the control unit controls the switch to connect the first electrode and the second electrode to the second circuit during the first period, and connect the first electrode and the second electrode to the first circuit during the second period in which a direct current is supplied.

4. The transdermal drug administration device according to claim 1, wherein the control unit changes the value of a direct current to be supplied between the first electrode and the second electrode depending on the obtained capacitance to keep the density of the direct current to be supplied to the external conductor constant.

5. The transdermal drug administration device according to claim 1, wherein the control unit stops the supply of direct current between the first electrode and the second electrode if the obtained capacitance is equal to or lower than a predetermined value.

6. The transdermal drug administration device according to claim 1, wherein the control unit issues an alert if the obtained capacitance is equal to or lower than a predetermined value.
